(19) United States
(12) Patent Application Publication
(10) Pub. No.: US 2009/0054947 A1
(43) Pub. Date: Feb. 26, 2009

(54) ELECTRODE CONFIGURATIONS FOR DIRECTIONAL LEADS

(75) Inventors: David Wayne Bourn, Maple Grove, MN (US); John L. Sommer, Coon Rapids, MN (US); Mark T. Marshall, Forest Lake, MN (US); Michael D. Eggen, Lake Elmo, MN (US); Gabriela C. Miyazawa, Fridley, MN (US)

Correspondence Address: Medtronic, Inc., 710 Medtronic Parkway, Minneapolis, MN, 55432, US

(73) Assignee: Medtronic, Inc., Minneapolis, MN (US)

(21) Appl. No.: 12/195,277

(22) Filed: Aug. 20, 2008

Related U.S. Application Data
(60) Provisional application No. 61/049,232, filed on Apr. 30, 2008.
(60) Provisional application No. 60/956,832, filed on Aug. 20, 2007.
(60) Provisional application No. 60/956,868, filed on Aug. 20, 2007.

(51) Int. Cl. A61N 1/362; A61N 1/05

(52) U.S. Cl. 607/30; 607/9

(57) ABSTRACT

A system includes an implantable electrical stimulation lead configured for intravenous introduction into a vessel proximate to a heart and an electrical stimulator. The lead comprises a lead body and at least three electrode segments. The electrical stimulator is coupled to the electrode segments and configures a first of the electrode segments as a first anode, a second of the electrode segments as a cathode, and a third of the electrode segments as a second anode, and delivers electrical stimulation to the heart via the cathode and first and second anodes. Additional techniques for delivering electrical stimulation include using multiple electrode segments as cathodes and electrically isolating other electrode segments. Other examples are directed to techniques for directing electrical therapy to a vagus nerve of a patient.

This application claims the benefit of U.S. Provisional Application No. 60/956,832, filed Aug. 20, 2007, U.S. Provisional Application No. 60/956,868, filed Aug. 20, 2007 and U.S. Provisional Application No. 61/049,232, filed Apr. 30, 2008, each of which are hereby incorporated by reference.

### TECHNICAL FIELD

The present disclosure relates to medical devices, more particularly to delivery of electrical stimulation via implantable medical leads.

### BACKGROUND

In the medical field, a wide variety of medical devices use implantable leads. For example, implantable cardiac pacemakers provide therapeutic stimulation to the heart by delivering pacing, cardioversion, or defibrillation pulses via implantable leads. Implantable cardiac pacemakers deliver such pulses to the heart via electrodes disposed on the leads, e.g., near distal ends of the leads. Implantable medical leads may be configured to allow electrodes to be positioned at desired cardiac locations so that the pacemaker can deliver pulses to the desired locations.

Implantable medical leads are also used with other types of stimulators to provide, as examples, neurostimulation, muscular stimulation, or gastric stimulation to target patient tissue locations via electrodes on the leads and located within or proximate to the target tissue. As one example, one or more implantable medical leads may be positioned proximate to the vagus nerve for delivery of neurostimulation to the vagus nerve. Additionally, implantable medical leads may be used by medical devices for patient sensing and, in some cases, for both sensing and stimulation. For example, electrodes on implantable medical leads may detect electrical signals within a patient, such as an electrocardiogram, in addition to delivering electrical stimulation.

For delivery of cardiac pacing pulses to the left ventricle (LV), an implantable medical lead is typically placed through the coronary sinus and into a coronary vein. However, when located in the coronary sinus or a coronary vein, an LV lead may also be located near the phrenic nerve. Phrenic nerve stimulation is generally undesirable during LV pacing therapy. In some instances, the implantable lead may need to be specifically positioned to avoid phrenic nerve stimulation during LV pacing therapy, which may result in placing the electrodes of the LV lead at a non-optimal site for LV pacing.

In some cases, implantable medical leads with ring electrodes are used as an alternative to cuff electrodes for delivery of neurostimulation to the vagus nerve. However, when located near the vagus nerve, the implantable medical lead may also be located near neck muscles. Stimulation of neck muscles is generally undesirable during therapeutic vagal neurostimulation.

### SUMMARY OF DISCLOSURE

In general, the present disclosure is directed toward delivering electrical stimulation using electrode segments in an anodal shielding configuration. For example, an implantable medical device (IMD) may configure a first electrode segment of an electrical stimulation lead as a cathode and two adjacent electrode segments of the lead, which may be on opposite sides of the first electrode segment, as anodes. This configuration may be referred to as an “anodal shielding” configuration in the sense that the anodes act as a shield around the cathode to substantially prevent propagation of the electrical field from the cathode to tissue that is beyond the anodes, e.g., tissue on an opposite side of the anode than the cathode. Anodal shielding may focus the electrical field propagating from the lead in a particular transverse direction relative to a longitudinal axis of the lead. Anodal shielding may also focus the electrical field propagating from the lead at a particular longitudinal direction. In this manner, anodal shielding may be useful in directing a stimulation field toward a target site and/or away from an undesirable site.

In one example, a system includes an implantable electrical stimulation lead configured for intravenous introduction into a vessel proximate to a heart. The lead comprises a lead body and at least three electrode segments. The system includes a cardiac stimulator coupled to the electrode segments. The electrical stimulator configures a first of the electrode segments as a first anode, a second of the electrode segments as a cathode, and a third of the electrode segments as a second anode, and delivers electrical stimulation to the heart via the cathode and first and second anodes.

In a different example, a system includes an implantable electrical therapy lead configured for implantation proximate to a vagus nerve of a patient. The lead comprises a lead body and at least three electrode segments. The system also includes a neurostimulator coupled to the electrode segments. The electrical stimulator configures a first of the electrode segments as a first anode, a second of the electrode segments as a cathode, and a third of the electrode segments as a second anode, and delivers electrical stimulation to the vagus nerve via the cathode and first and second anodes.

In another example, a method of delivering electrical stimulation to a heart comprises configuring a first electrode segment of an implantable electrical stimulation lead configured for intravenous introduction into a heart, as a first anode, a second electrode segment of the lead as a cathode, and a third electrode segment of the lead as a second anode; and delivering at least one electrical stimulation signal to the heart via the first, second, and third electrode segments.

In another example, a method of delivering electrical therapy to a vagus nerve of a patient comprises configuring a first electrode segment of an implantable electrical therapy lead configured for implantation proximate to the vagus nerve, as a first anode, a second electrode segment of the lead as a cathode, and a third electrode segment of the lead as a second anode; and delivering at least one electrical therapy signal to the vagus nerve via the first, second, and third electrode segments.

In another example, a system comprises means for configuring a first electrode segment of an implantable electrical stimulation lead as a first anode, a second electrode segment of the lead as a cathode, and a third electrode segment of the lead as a second anode; and means for delivering a stimulation signal via the first, second, and third electrode segments to one of a group consisting of a heart and a vagus nerve of a patient.

In a different example, a system comprises an implantable electrical stimulation lead configured for intravenous introduction into a vessel proximate to a heart. The lead comprises a lead body, a segmented electrode including at least three electrode segments, and insulative material between the at least three electrode segments at an outer circumference of the lead body at the segmented electrode. The at least three electrode segments are spaced apart circumferentially and separated by the insulative material such that the at least three electrode segments cover no more than about 270 degrees of the outer circumference of the lead body at the segmented electrode. The system further comprises a cardiac stimulator electrically coupled to the electrode segments.

In another example, a method of delivering electrical stimulation to a heart comprises configuring at least two adjacent electrode segments of a segmented electrode as cathodes, wherein the segmented electrode is included in an implantable electrical stimulation lead configured for intravenous introduction into a heart, configuring at least a third electrode segment of the segmented electrode to be electrically isolated from the cathodes, and delivering at least one electrical stimulation signal to the heart via the at least two adjacent electrode segments. The electrode segments of the segmented electrode are spaced apart circumferentially and separated by an insulative material such that the electrode segments of the segmented electrode cover no more than about 270 degrees of an outer circumference of the lead body at the segmented electrode.

In another example, a method of delivering electrical therapy to a vagus nerve of a patient comprises configuring at least two adjacent electrode segments of an implantable electrical stimulation lead configured for intravenous introduction into a heart as cathodes, configuring at least a third electrode segment of the lead to be electrically isolated from the cathodes, and delivering at least one electrical therapy signal to the vagus nerve via the at least two adjacent electrode segments. The electrode segments of the segmented electrode are spaced apart circumferentially and separated by an insulative material such that the electrode segments of the segmented electrode cover no more than about 270 degrees of an outer circumference of the lead body at the segmented electrode.

Electrode configuration in a directional lead may be particularly useful in left ventricle (LV) pacing applications. An IMD may configure electrodes segments of a lead in an anodal shielding configuration to direct the electrical field toward the myocardium and away from the phrenic nerve. Directing the electrical field towards the myocardium may reduce the amount of energy required for tissue capture of the myocardium for pacing therapies and, consequently, increase battery life. In addition, directing the electrical stimulation field towards the myocardium may reduce the likelihood of phrenic nerve stimulation, because the electrical stimulation field will generally be directed away from the phrenic nerve.

As another example, electrode configuration in a directional lead may be useful in stimulation of the vagus nerve. The vagus nerve is positioned proximate to muscles of the neck, which may inadvertently be stimulated along with the vagus nerve. Anodal shielding may control the direction and extent of propagation of the electrical field and aid in preventing stimulation of the neck muscles.

The electric fields produced using at least two adjacent electrode segments as cathodes may be combined with the techniques utilizing anodal shielding. A single IMD may optionally configure electrode segments using a single electrode segment as a cathode, using multiple electrode segments as cathodes, as well configuring electrode segments in anodal shielding configuration. An IMD that provides each of these techniques may be able to more successfully direct a stimulation field toward a target site and/or away from an undesirable site.

The details of one or more examples of the present disclosure are set forth in the accompanying drawings and the description below. Other features, objects, and benefits of the present disclosure will be apparent from the description and drawings, and from the claims.

### BRIEF DESCRIPTION OF DRAWINGS

FIG. 1 is a conceptual diagram illustrating an example implantable medical device (IMD) system.

FIG. 2 is a functional block diagram of an example of an IMD.

FIG. 3 is a functional block diagram of an example of a programmer for an IMD.

FIG. 4A is a side view of a distal end of a lead including electrode segments at its distal tip.

FIGS. 4B-4D are cross-sectional views of electrode segments at the distal tip of the lead of FIG. 4A and an electrical field propagating directionally from the electrode segments.

FIG. 5A is a side view of a distal end of another example of a lead including electrode segments at its distal tip.

FIG. 5B is a cross-sectional views of electrode segments at the distal tip of the lead of FIG. 5A.

FIG. 6 is a side view of a distal end of an example of a lead including a recessed electrode.

FIG. 7 is a side view of a distal end of an example of a lead including a protruded electrode.

FIG. 8 is a side view of a distal end of another example of a lead including electrode segments at its distal end.

FIG. 9 is a flow diagram illustrating a method of delivering stimulation therapy using an anodal shielding configuration.

FIG. 10 is a cross-section of a segmented lead used for experimentation.

FIG. 11 is a bar graph illustrating cardiac pacing and phrenic nerve capture thresholds determined by experimentation for various unipolar electrode configurations.

FIG. 12 is a bar graph illustrating cardiac pacing and phrenic nerve capture thresholds determined by experimentation for various anodal shielding electrode configurations.

FIGS. 13A-16B illustrate side and cross-section views of example leads having electrode segments, and example electrical fields produced when two of the electrode segments are charged.

FIG. 17 is a flow diagram illustrating a method of delivering stimulation therapy using an a pair of adjacent.

### DETAILED DESCRIPTION

While the description primarily refers to implantable medical leads and implantable medical devices, such as pacemakers and pacemaker-cardioverter-defibrillators, that deliver stimulation therapy to a patient's heart, the features and techniques described herein are useful in other types of medical device systems, which may include other types of implantable medical leads and implantable medical devices. For example, the features and techniques described herein may be used in systems with medical devices that deliver neurostimulation to the vagus nerve. As other examples, the features and techniques described herein may be embodied in systems that deliver other types of neurostimulation therapy (e.g., spinal cord stimulation or deep brain stimulation), stimulation of one or more muscles or muscle groups, stimulation of one or more organs such as gastric system stimulation, stimulation concomitant to gene therapy, and, in general, stimulation of any tissue of a patient.

In addition, while the examples shown in the figures include leads coupled at their proximal ends to a stimulation therapy controller, e.g., implantable medical device, located remotely from the electrodes, other configurations are also possible and contemplated. In some examples, a lead comprises a portion of a housing, or a member coupled to a housing, of stimulation generator located proximate to or at the stimulation site, e.g., a microstimulator. In other examples, a lead comprises a member at stimulation site that is wirelessly coupled to an implanted or external stimulation controller or generator. For this reason, as referred to herein, the term of a “lead” includes any structure having one or more stimulation electrodes disposed on its surface.

The techniques described herein are not limited to use with pacemakers, cardioverters or defibrillators. For example, leads including the features described herein may be used to deliver neurostimulation therapy from a medical device to target neural tissues of a patient, such as the vagal nerve. Furthermore, although described herein as being coupled to IMDs, implantable medical leads of according to the present disclosure may also be percutaneously coupled to an external medical device for deliver of electrical stimulation to target locations within the patient. Additionally, the described techniques are not limited to examples that deliver electrical stimulation to a patient, and are also applicable to examples in which electrical signals or other physiological parameters are sensed via one or more electrodes of an implantable medical lead.

For example, for effective cardiac pacing, stimulation therapy can be of adequate energy for a given location to cause depolarization of the myocardium. Sensing a physiological parameter of the patient may be used to verify that pacing therapy has captured the heart, i.e., caused depolarization of the myocardium, to initiate a desired response to the therapy such as, for example, providing pacing, resynchronization, defibrillation and/or cardioversion. Such sensing may include sensing an evoked R-wave or P-wave after delivery of pacing therapy, sensing for the absence of an intrinsic R-wave or P-wave prior to delivering pacing therapy, or detecting a conducted depolarization in an adjacent heart chamber.

These and other physiological parameters may be sensed using electrodes that may be also used to deliver stimulation therapy. For example, a system may sense physiological parameters using the same electrodes used for providing stimulation therapy or electrodes that are not used for stimulation therapy. As with stimulation therapy, selecting which electrode(s) are used for sensing physiological parameters of a patient may alter the signal quality of the sensing techniques. For this reason, sensing techniques may include one or more algorithms to determine the suitability of each electrode or electrode combination in the stimulation therapy system for sensing one or more physiological parameters. Sensing physiological parameters may also be accomplished using electrode or sensors that are separate from the stimulation electrodes, e.g., electrodes capable of delivering stimulation therapy, but not selected to deliver the stimulation therapy that is actually being delivered to the patient.

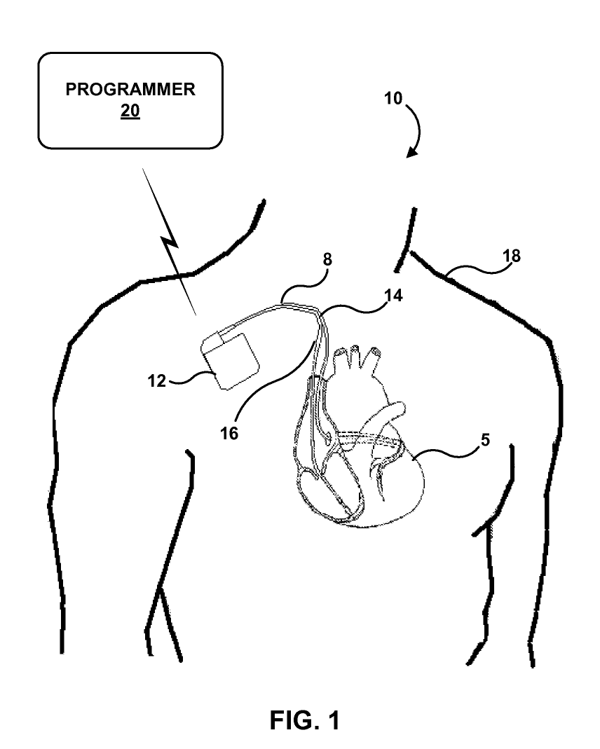

FIG. 1 is a conceptual diagram illustrating an example implantable medical system 10 comprising implantable medical device (IMD) 12 and implantable medical leads 14, 16 electrically coupled to IMD 12. In the example shown in FIG. 1, system 10 is implanted to deliver stimulation therapy to heart 5 of patient 18. Patient 18 ordinarily, but not necessarily, will be a human patient.

In the example shown in FIG. 1, IMD 12 is a cardiac pacemaker, cardioverter, defibrillator, or pacemaker-cardioverter-defibrillator (PCD) that generates therapeutic electrical stimulation for pacing, cardioversion or defibrillation, which may take the form of pulses or continuous time signals. Leads 14, 16 each include at least one electrode that are each positioned within (e.g., intravenously) or proximate to (e.g., epicardially) heart 5 in order to deliver the therapeutic electrical stimulation from IMD 12 to heart 5. In some examples, at least one of leads 14, 16 may provide stimulation to heart 5 without contacting heart 5, e.g., at least one of leads 14, 16 may include a subcutaneous electrode.

In the illustrated example, a distal end of lead 14 is positioned proximate to the left ventricle of patient 18 and, more particularly, within the coronary sinus or a coronary vein accessed via the coronary sinus. Lead 14 is configured for intravenous introduction into heart 5. For example, lead 14 may have a lead body diameter of between 0.020 inches and 0.100 inches. Distal end of lead 16 is positioned within the right ventricle of patient 18. Accordingly, in the illustrated example, lead 14 may be referred to as a left ventricular (LV) lead, and lead 16 may be referred to as a right ventricular (RV) lead. IMD 12 may deliver coordinated pacing signals to heart 5 via leads 14 and 16 to, for example, to resynchronize the action of the left and right ventricles.

As shown in FIG. 1, system 10 may also include a programmer 19, which may be a handheld device, portable computer, or workstation that provides a user interface to a clinician or other user. The clinician may interact with the user interface to program stimulation parameters for IMD 12, which may include, for example, the electrodes of leads 14, 16 that are activated, the polarity of each of the activated electrodes, a current or voltage amplitude for each of the activated electrodes and, in the case of stimulation in the form of electrical pulses, pulse width and pulse rate (or frequency) for stimulation signals to be delivered to patient 18. As referred to herein, an amplitude of stimulation therapy may be characterized as a magnitude of a time varying waveform. For example, an amplitude of stimulation therapy may be measured in terms of voltage (volts), current (ampere), or electric field (volts/meter). Typically, amplitude is expressed in terms of a peak, peak to peak, or root mean squared (rms) value.

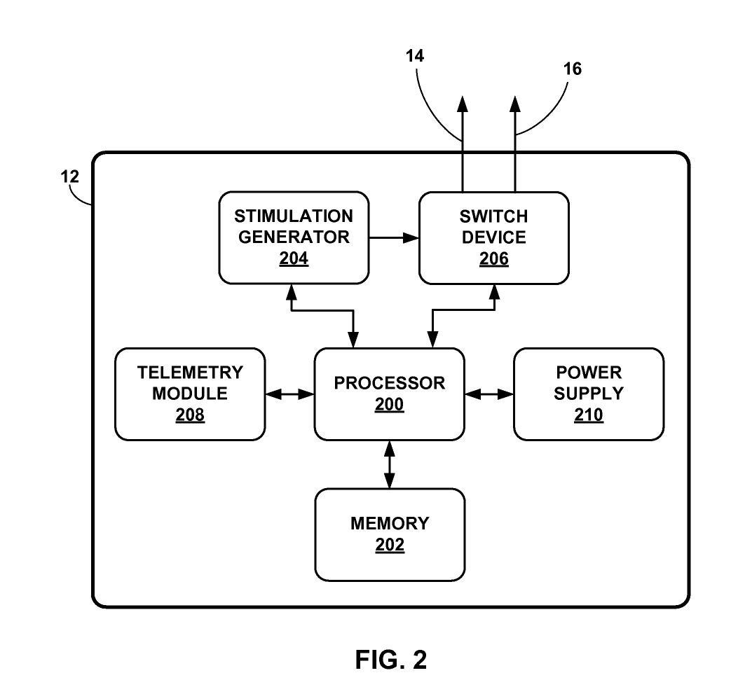

FIG. 2 is a functional block diagram of an example of IMD 12. IMD 12 includes a processor 200, memory 202, stimulation generator 204, switch device 206, telemetry module 208, and power source 210. As shown in FIG. 2, switch device 206 is coupled to leads 14, 16. Alternatively, switch device 206 may be coupled to a single lead or more than two leads directly or indirectly (e.g., via a lead extension, such as a bifurcating lead extension that may electrically and mechanically couple to two leads) as needed to provide stimulation therapy to patient 12.

Memory 202 includes computer-readable instructions that, when executed by processor 200, cause IMD 12 to perform various functions. Memory 202 may include any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital media.

Stimulation generator 204 produces stimulation signals (e.g., pulses or continuous time signals, such as sine waves) for delivery to patient 18 via selected combinations of electrodes carried by leads 14, 16. Processor 200 controls stimulation generator 204 to apply particular stimulation parameters specified by one or more of programs (e.g., programs stored within memory 222), such as amplitude, pulse width, and pulse rate. Processor 200 may include a microprocessor, a controller, a digital signal processor (DSP), an application specific integrated circuit (ASIC), a field-programmable gate array (FPGA), or equivalent discrete or integrated logic circuitry.

Processor 200 also controls switch device 206 to apply the stimulation signals generated by stimulation generator 204 to selected combinations of the electrodes of leads 14, 16 with a polarity as specified by one or more stimulation programs. In particular, switch device 206 couples stimulation signals to selected conductors within leads 14, 16 which, in turn, delivers the stimulation signals across selected electrodes of leads 14, 16. Switch device 206 may be a switch array, switch matrix, multiplexer, or any other type of switching device suitable to selectively couple stimulation energy to selected electrodes. Hence, stimulation generator 204 is coupled to the electrodes of leads 14, 16 via switch device 206 and conductors within leads 14, 16.

Stimulation generator 204 may be a single- or multi-channel stimulation generator. In particular, stimulation generator 204 may be capable of delivering, a single stimulation pulse, multiple stimulation pulses, or a continuous signal at a given time via a single electrode combination or multiple stimulation pulses at a given time via multiple electrode combinations. In some examples, multiple channels of stimulation generator 204 may provide different stimulation signals, e.g., pulses, to different electrodes at substantially the same time. For example, multiple channels of stimulation generator 204 may provide signals with different amplitudes to different electrodes at substantially the same time.

Telemetry module 208 supports wireless communication between IMD 12 and an external programmer 19 or another computing device under the control of processor 200. Processor 200 of IMD 14 may receive, as updates to programs, values for various stimulation parameters such as amplitude and electrode combination, from programmer 19 via telemetry interface 208. The updates to the therapy programs may be stored within memory 202.

The various components of IMD 14 are coupled to power supply 210, which may include a rechargeable or non-rechargeable battery. A non-rechargeable battery may be selected to last for several years, while a rechargeable battery may be inductively charged from an external device, e.g., on a daily or weekly basis. In other examples, power supply 210 may be powered by proximal inductive interaction with an external power supply carried by patient 18.

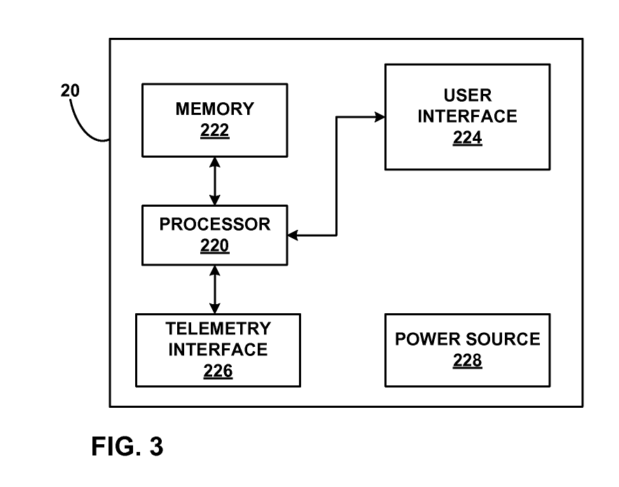

FIG. 3 is a functional block diagram of an example of programmer 19. As shown in FIG. 3, external programmer 19 includes processor 220, memory 222, user interface 224, telemetry module 226, and power source 228. A clinician or another user may interact with programmer 19 to generate and/or select therapy programs for delivery in IMD 12. For example, in some examples, programmer 19 may allow a clinician to define stimulation fields, e.g., select appropriate stimulation parameters for one or more stimulation programs to define the desired or optimal stimulation field. Programmer 19 may be used to select stimulation programs, generate new stimulation programs, and transmit the new programs to IMD 12. Processor 220 may store stimulation parameters as one or more stimulation programs in memory 222. Processor 220 may send programs to IMD 12 via telemetry module 226 to control stimulation automatically and/or as directed by the user.

Programmer 19 may be one of a clinician programmer or a patient programmer, i.e., the programmer may be configured for use depending on the intended user. A clinician programmer may include more functionality than the patient programmer. For example, a clinician programmer may include a more featured user interface, allow a clinician to download therapy usage, sensor, and status information from IMD 12, and allow a clinician to control aspects of IMD 12 not accessible by a patient programmer example of programmer 19.

A user, either a clinician or patient 12, may interact with processor 220 through user interface 224. User interface 224 may include a display, such as a liquid crystal display (LCD), light-emitting diode (LED) display, or other screen, to show information related to stimulation therapy, and buttons or a pad to provide input to programmer 19. Buttons may include an on/off switch, plus and minus buttons to zoom in or out or navigate through options, a select button to pick or store an input, and pointing device, e.g. a mouse, trackball, or stylus. Other input devices may be a wheel to scroll through options or a touch pad to move a pointing device on the display. In some examples, the display may be a touch screen that enables the user to select options directly from the display screen.

Programmer 19 may be a handheld computing device, a workstation or another dedicated or multifunction computing device. For example, programmer 19 may be a general purpose computing device (e.g., a personal computer, personal digital assistant (PDA), cell phone, and so forth) or may be a computing device dedicated to programming IMD 12.

Processor 220 processes instructions from memory 222 and may store user input received through user interface 224 into the memory when appropriate for the current therapy. Processor 220 may comprise any one or more of a microprocessor, digital signal processor (DSP), application specific integrated circuit (ASIC), field-programmable gate array (FPGA), or other digital logic circuitry.

Memory 222 may include instructions for operating user interface 224, telemetry module 226 and managing power source 228. Memory 222 may store program instructions that, when executed by processor 220, cause the processor and programmer 19 to provide the functionality ascribed to them herein. Memory 222 may include any one or more of a random access memory (RAM), read-only memory (ROM), electronically-erasable programmable ROM (EEPROM), flash memory, or the like.

Wireless telemetry in programmer 19 may be accomplished by radio frequency (RF) communication or proximal inductive interaction of programmer 19 with IMD 12. This wireless communication is possible through the use of telemetry module 226. Accordingly, telemetry module 226 may include circuitry known in the art for such communication.

Power source 228 delivers operating power to the components of programmer 19. Power source 228 may include a battery and a power generation circuit to produce the operating power. In some examples, the battery may be rechargeable to allow extended operation. Recharging may be accomplished through proximal inductive interaction, or electrical contact with circuitry of a base or recharging station. In other examples, primary batteries may be used. In addition, programmer 19 may be directly coupled to an alternating current source; such would be the case with some computing devices, such as personal computers.

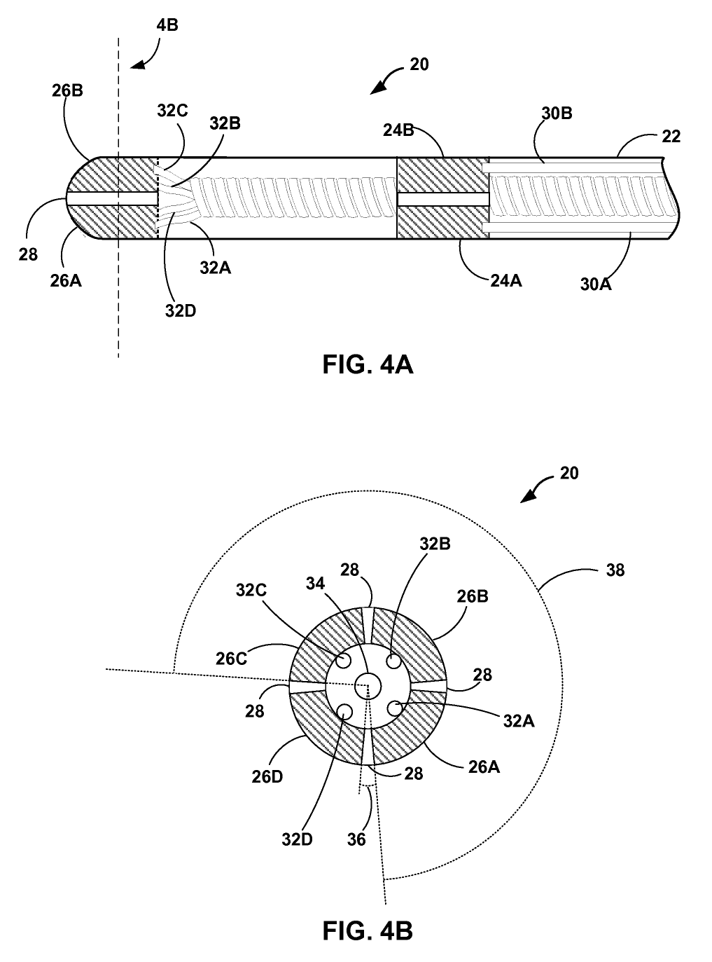

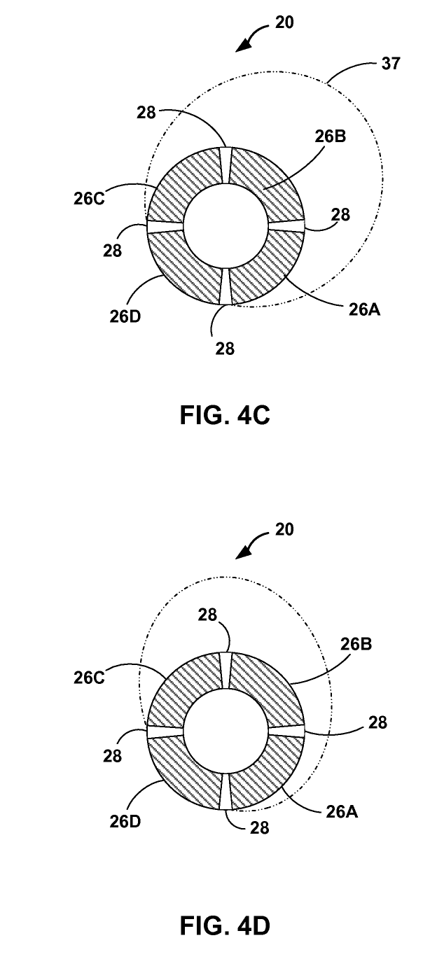

FIG. 4A is a side view of a distal end of an example of a lead 20, which may, for example, correspond to either of leads 14, 16 of FIG. 1. A proximal end (not shown) of lead 20 may be coupled to an IMD (e.g., IMD 12 of FIG. 1). Lead 20 includes a lead body 22, electrodes 24A, 24B, and 26A-26D (electrodes 26C and 26D are not shown in FIG. 4A), and one or more elongated conductors (not shown) electrically coupled to the electrodes and covered or surrounded by one or more elongated insulative bodies. Electrodes 24A, 24B, and 26A-26D are exposed to tissue of the patient, which allows data to be sensed from the tissue and/or therapy delivered to the patient.

Lead body 22 may be formed from a biocompatible material. Exemplary biocompatible material includes one or more of polyurethane, silicone, and fluoropolymers such as tetrafluroethylene (ETFE), polytetrafluroethylene (PTFE), and/or expanded PTFE (i.e. porous ePTFE, nonporous ePTFE).

As shown in FIG. 4A, electrodes 24A and 24B are flush or isodiametric with lead body 22 and may be segmented or partial ring electrodes, each of the electrode segments 24A and 24B extending along an arc less than 360 degrees (e.g., 90-120 degrees). Segmented or partial ring electrodes may be useful for providing an electrical stimulation field that is predominantly focused in a particular transverse direction relative to the longitudinal axis of lead 20, and/or targeting a particular stimulation site. In other examples, instead of or in addition to electrodes 24A and 24B, lead 20 may include a ring electrode extending substantially around the entire periphery, e.g., circumference, of lead 20.

In the illustrated example, electrodes 26A-26D are also segmented or partial ring electrodes, which do not extend substantially around the entire periphery of the lead body 22. Electrodes 26C and 26D are located on the circumferential portion of lead body 22 not visible in FIG. 4A. As described in further detail below, FIG. 4B is a cross-sectional view of electrodes 26A-26D along line 4B in FIG. 4A, and illustrates the approximate locations of electrodes 26C and 26D. Electrodes 26A-26D may, but need not be, located at the same axial position along the length of lead body 22. When electrodes 26A-26D are located at the same axial position of lead body 22, electrodes 24A-24D may form a row of electrode segments. In some examples, electrodes 26A-26D may be evenly spaced around the periphery of lead 20. Additionally, each of individual electrode segments 26A-26D may be separated by insulative material 28, which may aid in electrically isolating each of electrodes 26A-26D.

Each of electrodes 24A, 24B, and 26A-26D can be made from an electrically conductive, biocompatible material, such as platinum iridium. In addition, one or more of electrodes 24A, 24B, and 26A-26D may function as sensing electrodes. Sensing electrodes can continuously or periodically send one or more signals through lead 20 to processor 200. Electrical signals from sensing electrodes typically include physiological data related to patient 18 (FIG. 1). Exemplary physiological data includes an electrocardiogram (ECG), heart rate, QRS width, atrioventricular (AV) Dissociation, respiration rate, respiratory volume, core temperature, diaphragmatic stimulation, skeletal muscle activity, blood oxygen level, cardiac output, blood pressure, intercardiac pressure, time derivative of intercardiac pressure (dP/dt), electromyogram (EMG) parameters, an electroencephalogram (EEG) parameters and physiological data.

The configuration, type, and number of electrodes 24A, 24B, and 26A-26D are merely exemplary. In other examples, lead 20 may include any configuration, type, and number of electrodes 24A, 24B, and 26A-26D, and is not limited to the example illustrated in FIGS. 4A and 4B.

Within lead body 22, lead 20 also includes electrical conductors 30A and 30B coupled to electrodes 24A and 24B, and electrical conductors 32A-32D coupled to electrode segments 26A-26D, respectively. In the illustrated example, conductors 32A-32D are coiled along the length of lead body 22 (e.g., in a multiconductor coil), and conductors 30A and 30B lie axial to conductors 32A-32D. Conductors 30A and 30 B may or may not be coiled. In the example illustrated in FIG. 4A, each of conductors 30A, 30B, and 32A-32D is electrically coupled to a single one of electrodes 24A, 24B, and 26A-26D, respectively. In this manner, each of electrodes 24A, 24B, and 26A-26D may be independently activated. In other examples, a lead including multiple electrodes may include a multiplexer or other switching device such that the lead may include fewer conductors than electrodes, while allowing each of the electrodes to be independently activated. The switching device may be responsive to commands from the IMD or an external source to selectively couple the electrodes to the conductors for delivery of stimulation or for sensing.

The configuration, type, and number of conductors 30A, 30B, and 32A-32D is not limited to the example illustrated in FIG. 4A and, in other examples, lead 20 may include any configuration, type, and number of conductors. As one example, in some examples, each of conductors 30A, 30B, and 32A-32D may be coiled conductors. Additionally or alternatively, one conductor may be electrically coupled to two or more electrodes.

FIG. 4B is a cross-sectional view of electrode segments 26A-26D along line 4B in FIG. 4A. As previously described, each of electrode segments 26A-26D is separated by insulative material 28. The center of lead 20 may include a lumen 34 to accommodate a delivery device such as a stylet, guidewire or a hybrid of a stylet and guidewire. A delivery device may be used to help position lead 20 at a target location during implantation of lead 20. Electrical conductors 32A-32D are coupled to electrode segments 26A-26D, respectively. Each of conductors 32A-32D extends from electrodes 26A-26D to a proximal end of lead body 22 to couple electrodes 26A-26D to an IMD (e.g., IMD 12 of FIG. 1).

Electrode segments 26A-26D may be useful in directing a stimulation field toward a target site and/or away from an undesirable site. For example, one or more of electrode segments 26A-26D may be activated (e.g., as a cathode or an anode) to deliver stimulation to patient 18 (FIG. 1). As will be described in greater detail below, the direction of the stimulation field, e.g., the radial direction relative to the longitudinal axis of elongated lead body 22 or “side” of the lead on which the field is present, may be based on which of electrode segments 26A-26D are activated. Electrodes 24A and 24B may further aid in steering the stimulation field in a particular direction, e.g., longitudinal direction, and/or sensing a patient condition on a particular side of lead body 22. Additionally, a current or voltage amplitude may be selected for each of the active electrodes. During movement of lead 20, one or more of the electrodes may produce different amplitudes to further aid in controlling the direction of the stimulation field. In one embodiment of a system having two anodes with different amplitudes, each anode adjacent to a cathode, generally, the stimulation field is at least partially biased towards the anode with the higher current or voltage amplitude. As one example, a directional stimulation field may be particularly useful in left ventricle (LV) pacing applications. An IMD (e.g., IMD 12 of FIG. 1) may configure electrodes 24A, 24B, and 26A-26D to direct the stimulation field toward the myocardium and away from the phrenic nerve. More specifically, when lead 20 is transvenously placed proximate to the LV of patient 18 (FIG. 1), it may be desirable to only activate one or more of electrodes 24A, 24B, and 26A-26D positioned proximate to the myocardium (e.g., facing or in contact with the myocardium) rather than those proximate to the epicardium. Selectively activating one or more of electrodes 24A, 24B, and 26A-26C to direct the electrical stimulation field towards the myocardium may reduce the amount of energy required for tissue capture of the myocardium for pacing therapies and, consequently, increase battery life. In addition, directing the electrical stimulation field towards the myocardium may reduce the likelihood of phrenic nerve stimulation, because the electrical stimulation field will generally be directed away from the phrenic nerve. In other words, when the electrical stimulation field is directed toward the myocardium, the excess electrical field directed away from the myocardium and across the pericardium where the phrenic nerve lies that may be present when the electrical stimulation is delivered via a ring electrode that extends substantially completely around the circumference or periphery of a lead may be reduced or eliminated.

A directional stimulation field may be particularly useful when phrenic nerve stimulation occurs post-implant. Using a conventional LV lead, when phrenic nerve stimulation occurs post-implant, the clinician may need to either extract the lead to reposition it or abandon LV pacing. Using a lead with electrode segments, the clinician may alter the electrode configuration, e.g., by selecting a different combination of electrode segments or altering the relative amplitudes of stimulation delivered by active electrode segments, to aid in directing the stimulation field away from the phrenic nerve.

As another example, a directional stimulation field may be useful in stimulation of the vagus nerve. Stimulation of the vagus nerve may be performed to decrease heart rate. The vagus nerve is positioned proximate to muscles of the neck, which may inadvertently be stimulated along with the vagus nerve. Controlling the direction of propagation of the stimulation field may aid in preventing stimulation of the neck muscles. As another example, a directional electrical field may be useful in atrial stimulation where it may be desirable to avoid stimulating specific ischemic tissue regions which may result in an arrhythmia. In general, electrodes segments 24A, 24B, and 26A-26D may be useful in any application where controlling the direction of propagation of the stimulation field is desirable.

In one example, the IMD (e.g., IMD 12 of FIG. 1) may configure a first electrode segment as a cathode and two adjacent electrode segments, which may be on opposite sides of the first electrode segment, as anodes. This configuration may be referred to as an “anodal shielding” configuration in the sense that the anodes act as a shield around the cathode to substantially prevent propagation of the electrical field from the cathode to tissue that is beyond the anodes, e.g., tissue on an opposite side of the anode than the cathode.

For example, IMD 12 may configure electrode segment 26B as a cathode and adjacent electrodes segments 26A and 26C on opposite sides of electrode segment 26B as anodes. Electrode segments 26A and 26C (the anodes) may substantially constrain the electrical field propagating from electrode segment 26B (the cathode) to the side or angular section of lead 38 that includes electrode segment 26B. The electrical field may be centered between electrode segments 26A and 26C and, depending on the stimulation amplitudes for each of electrode segments 26A-26C, may be centered substantially over electrode segment 26B. IMD 12 may activate electrode segments 26A-26D in different configurations and different amplitudes based on the desired direction of the stimulation field. One or more of electrode segments 24A and 24B may additionally or alternatively be activated as an anode or cathode to aid in controlling the direction of propagation of the stimulation field.

Anodal shielding may limit the size of the stimulation field. For example, the anodes may determine the extent and shape of a volume of tissue to which the stimulation field propagates. In some examples, an anodal shielding configuration may prevent the stimulation field from extending past the anodes. While the current example of anodal shielding only includes a single electrode configured as a cathode, anodal shielding may also include configuring multiple electrodes, e.g., adjacent electrodes, as cathodes.

The spacing between each of electrode segments 26A-26D may also influence the size of the stimulation field. In the example illustrated in FIG. 4B, electrodes 26A-26D are evenly or about evenly spaced around the periphery of lead 20 with arc 36 separating each of electrodes 26A-26D. Separation arc 36 may be selected based on the desired size of the stimulation field. In other examples, electrode segments 26A-26C may be unevenly spaced around the periphery of lead 20.

FIG. 4C is another cross-sectional view of electrode segments 26A-26D. FIG. 4C illustrates stimulation field 37 emanating from electrode segments 26A-26C. As described with respect to FIG. 4B, IMD 12 may configure electrode segment 26B as a cathode and adjacent electrodes segments 26A and 26C on opposite sides of electrode segment 26B as anodes. Electrode segments 26A and 26C (the anodes) may substantially constrain stimulation field 37 from propagating past electrode segments 26A and 26C (the anodes). In the example illustrated in FIG. 4C, stimulation field 37 is substantially centered over electrode segment 26B. IMD 12 may activate each of electrode segments 26A-26C with substantially the same amplitude to generate stimulation field 37 substantially centered over electrode segment 26B. For example, substantially similar voltage amplitudes may vary by no more than 0.1 volts, and substantially similar current amplitudes may vary by no more than 0.1 milliamps. IMD 12 may activate electrode segments 26A-26D in different configurations based on the desired direction of the stimulation field.

FIG. 4D is another cross-sectional view of electrode segments 26A-26D. FIG. 4D illustrates stimulation field 39 emanating from electrode segments 26A-26C. As described with respect to FIGS. 4B and 4C, IMD 12 may configure electrode segment 26B as a cathode and adjacent electrodes segments 26A and 26C on opposite sides of electrode segment 26B as anodes. Electrode segments 26A and 26C (the anodes) may substantially constrain stimulation field 37 from propagating past electrode segments 26A and 26C (the anodes). In the example illustrated in FIG. 4D, stimulation field 39 is skewed toward electrode 26C compared to stimulation field 37 of FIG. 4C. Rather than being substantially centered over electrode 26B (the central cathode), stimulation field 37 is shifted toward electrode 26C. IMD 12 may activate electrode segments 26A-26C with different current or voltage amplitudes to generate stimulation field 39 shifted toward electrode 26C. Additionally, IMD 12 may activate electrode segments 26A-26D in different configurations based on the desired direction of the stimulation field. For example, IMD 12 may selectively activate two electrode segments 26A-26D a bipolar configuration.

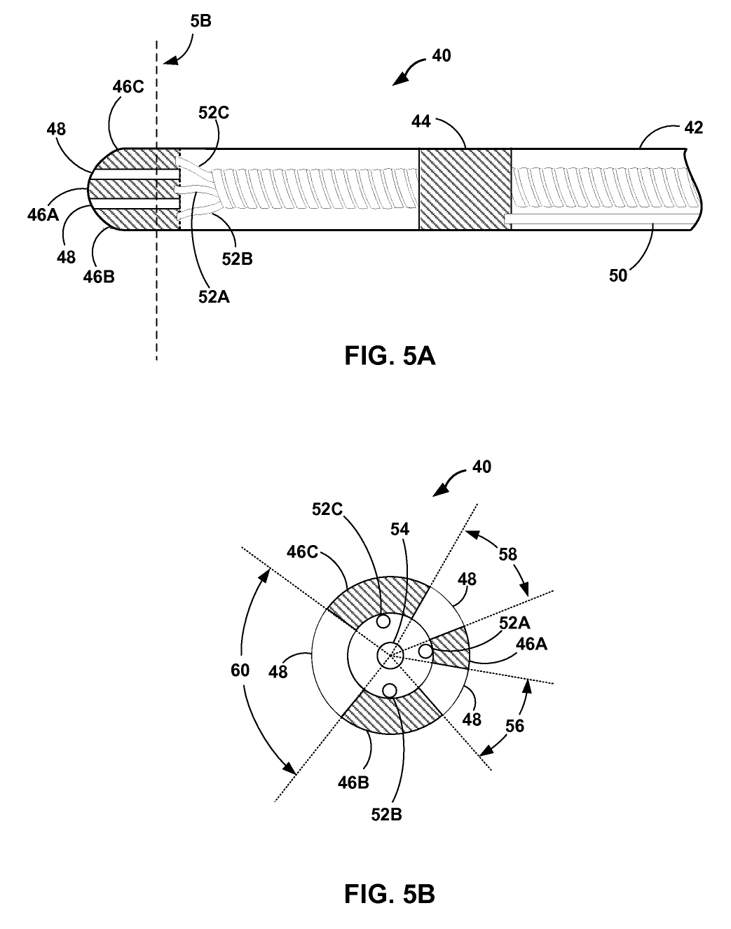

FIG. 5A is a side view of a distal end of another example of a lead 40. A proximal end (not shown) of lead 40 may be coupled to an IMD (e.g., IMD 12 of FIG. 1). Lead 40 includes a lead body 42 and electrodes 44 and 46A-46C. An outer surface of lead body 42 can be formed from a biocompatible material such as, for example, polyurethane or silicone. As shown in FIG. 5A, electrode 44 may be a ring electrode extending substantially around the entire periphery, e.g., circumference, of lead 40. In other examples, electrode 44 may comprise segmented or partial ring electrodes, each of the electrode segments extending along an arc less than 360 degrees (e.g., 90-120 degrees).

In the illustrated example, electrodes 46A-46C are electrode segments, which do not extend substantially around the entire periphery of the lead 40. Electrodes 46A-46C may, but need not be, located at the same axial position along the length of lead body 42. When electrodes 46A-46C are located at the same axial position of lead body 42, electrodes 46A-46C may form a row of electrode segments. In some examples, electrodes 46A-46C may be evenly spaced around the periphery of lead 40. In other embodiments, electrodes 46A-46C can be about evenly spaced around the periphery of lead 40. In still yet other embodiments, electrodes 46A-46C are unevenly spaced around the periphery of lead 40. Additionally, each of individual electrode segments 46A-46C may be separated by insulative material 48, which may aid in electrically isolating each of electrodes 46A-46C. Insulative material 48 is a biocompatible material having an impedance sufficient to prevent shorting between electrode segments during stimulation therapy. For example, insulative material 48 may comprise polyurethane, silicone, and fluoropolymers such as tetrafluroethylene (ETFE), polytetrafluroethylene (PTFE), and/or expanded PTFE (i.e. porous ePTFE, nonporous ePTFE).

Each of electrodes 44 and 46A-46C can be made from an electrically conductive, biocompatible material, such as platinum iridium. In addition, one or more of electrodes 44 and 46A-46C may function as sensing electrodes that monitor internal physiological signals of patient 18 (FIG. 1). The configuration, type, and number of electrode 44 and 46A-46C are merely exemplary. In other examples, lead 40 may include any configuration, type, and number of electrodes 44 and 46A-46C and is not limited to the example illustrated in FIG. 5A.

Electrode segments 46A-46C can be useful in directing a stimulation field toward a target site and/or away from an undesirable site. For example, one or more of electrode segments 46A-46C can be activated (e.g., as a cathode or an anode) to deliver stimulation to patient 18 (FIG. 1). The direction of the stimulation field may be based on which electrode segments 46A-46C are activated. A current or voltage amplitude can be selected for each of the active electrodes to further aid in controlling the direction of the stimulation field. Electrodes activated with unequal amplitudes may shift the direction of the stimulation field relative to a central position of a group of active electrodes, e.g., relative to a central cathode, such as described with respect to stimulation field 39 of FIG. 4D. For example, unequal voltage amplitudes may vary by at least about 0.1 volts, and unequal current amplitudes may vary by at least about 0.1 milliamps.

An IMD (e.g., IMD 12 of FIG. 1) may configure electrode segments 46A-46C in an anodal shielding configuration. For example, IMD 12 may configure electrode segment 46A as a cathode and electrode segments 46B and 46C on opposite sides of electrode segment 46A as anodes. Anodal shielding may limit the size of the stimulation field. For example, the anodes may determine the extent and shape of area that experiences the effect of the stimulation field. In some examples, an anodal shielding configuration may prevent the stimulation field from extending past the anodes.

Electrode 44 may allow a conventional electrode configuration, which may be used as an alternative to configurations including electrode segments 46A-46C. Conventionally, a LV lead may utilize a ring electrode as a cathode and the IMD (e.g., IMD 12 of FIG. 1) or a conductive portion (e.g., a coil electrode) on another lead (e.g., a lead with a distal end implanted in the right ventricle) as an anode in a unipolar configuration. As one example, a superior vena cava (SVC) coil and/or a right ventricle (RV) coil of a lead with a distal end implanted in the right ventricle may be activated as an anode. Electrode 44 may activated as cathode in a conventional unipolar configuration. Electrode 44 may provide a clinician with a familiar fall-back configuration.

Lead 40 also includes electrical conductor 50 coupled to electrode 44, and electrical conductors 52A-52C coupled to electrode segments 46A-46C, respectively. In the illustrated example, conductors 52A-52C are coiled along the length of lead body 42 (e.g., in a multiconductor coil), and conductor 50 lies axial to conductors 52A-52C. In the example illustrated in FIG. 5A, each of conductors 50 and 52A-52C is electrically coupled to a single one of electrodes 44 and 46A-46C, respectively. In this manner, each of electrodes 44 and 46A-46C may be independently activated. Electrodes 44 and 46A-46C may be coupled to an IMD (e.g., IMD 12 of FIG. 1) using an industry standard-4 (such as a IS-4) connector, which allows the connection of up to four independently activatable channels or other suitable connectors. More specifically, conductors 50 and 52A-52C may couple electrodes 44 and 46A-46C to an IMD (e.g., IMD 12 of FIG. 1) via an IS-4 connector. An IS-4 compatible lead may be easily coupled to an IMD configured according to the IS-4 standard.

The configuration, type, and number of conductors 50 and 52A-52C is not limited to the example illustrated in FIG. 5A and, in other examples, lead 40 may include any configuration, type, and number of conductors. As one example, in some examples, each of conductors 50 and 52A-52C may be coiled conductors. Additionally or alternatively, one conductor may be electrically coupled to two or more electrodes. In other examples, lead 40 may include a multiplexer such that lead body 42 may include fewer conductors than electrodes while allowing each of the electrodes to be independently activated.

FIG. 5B is a cross-sectional view of lead 40 taken through electrode segments 46A-46C. As previously described, each of electrode segments 46A-46C is separated by insulative material 48. The center of lead 40 may include a lumen 54 to accommodate a delivery device such as a stylet, guidewire or a hybrid of a stylet and guidewire. A delivery device may be used to help position lead 40 at a target location during implantation of lead 40. Electrical conductors 52A-52C are coupled to electrode segments 46A-46C, respectively. Each of conductors 52A-52C extends from electrodes 46A-46C to a proximal end of lead body 42 to couple electrodes 46A-46C to an IMD (e.g., IMD 12 of FIG. 1).

As described previously, the separation between electrode segments may impact the size of the stimulation field. In the example illustrated in FIG. 5B, electrodes 46A and 46B are separated by arc 56, electrodes 46A and 46C are separated by arc 58, and electrodes 46B and 46C are separated by arc 60. Each of arcs 56, 58, and 60 may extend anywhere from about 1 degree of arc to about 179 degrees of arc. In the example illustrated in FIG. 5B, arcs 56 and 58 are about the same size, and arc 60 is greater than each of arcs 56 and 58.

In some examples, electrodes 46A-46C may have different surface areas. For example, the surface area of the anode electrodes may be equal to or larger than the surface area of the cathode electrode. For purposes of example, electrode 46A may be referred to as cathode 46A and electrodes 46B and 46C may be referred to as anodes 46B and 46C. However, electrodes 46A-46C are not limited to this configuration.

In some examples, the ratio of the surface area of cathode 46A to the surface area of each of anodes 46B and 46C may range from about 1 to 1 to about 1 to 7. In some examples, the ratio of the surface area of cathode 46A to the surface area of each of anodes 46B and 46C may be about 1 to 3. Providing cathode 46A with a smaller surface area than the surface area of each of anodes 46B and 46C may limit anodal corrosion. Additionally, increasing the surface area of each of anodes 46B and 46C may spread the voltage drop out over the surface area of anodes 46B and 46C.

In one example, at least a portion of lead 40, such as electrodes 44 or a separate marker loaded in or formed on lead body 42, may include a radio-opaque material that is detectable by imaging techniques, such as fluoroscopic imaging or x-ray imaging. For example, as described previously, electrodes 44 and 46A-46C may be made of platinum iridium, which is detectable via imaging techniques. This feature may be helpful for maneuvering lead 40 relative to a target site within the body. Radio-opaque markers, as well as other types of markers, such as other types of radiographic and/or visible markers, may also be employed to assist a clinician during the introduction and withdrawal of stimulation lead 40 from a patient. Markers identifying the location of each electrode may be particularly helpful. Since the electrodes rotate with the lead body, a clinician may rotate the lead and the electric field to stimulate a desired tissue. Markers may help guide the rotation.

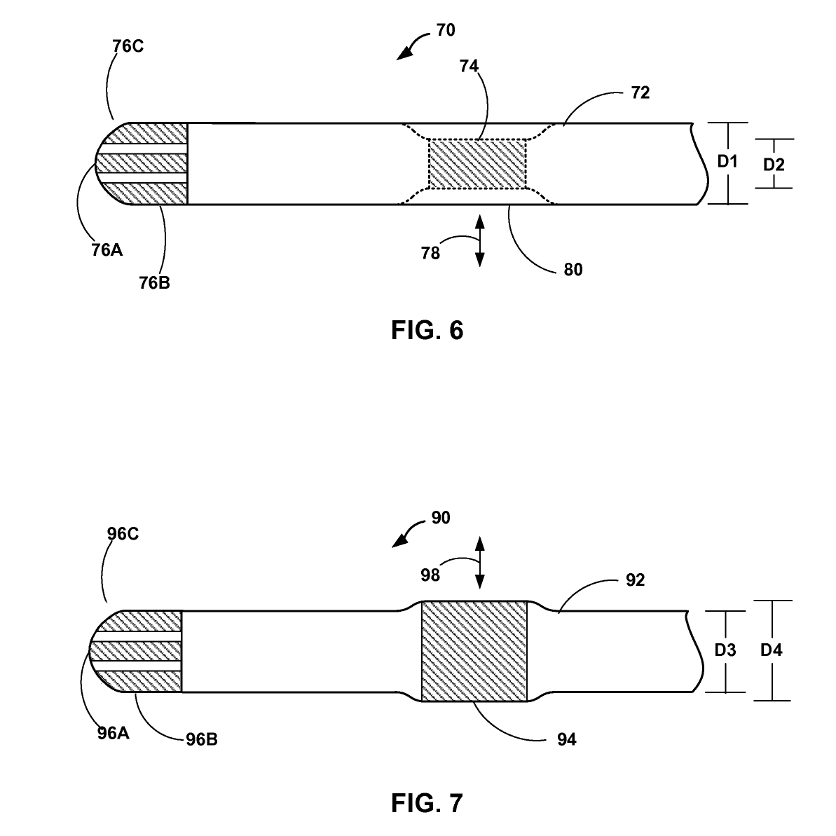

FIG. 6 is a side view of an example of a distal end of a lead 70. Lead 70 is substantially similar to lead 40 of FIGS. 5A and 5B but includes a recessed ring electrode 74. Lead 70 includes a lead insulative body 72 and electrodes 74 and 76A-76C. Electrodes 76A-76C may be substantially similar to electrodes 46A-46C of lead 40 and may be arranged in a similar configuration.

Electrode 74 is recessed relative to lead body 72. More particularly, the diameter D2 of electrode 74 is smaller than the diameter D1 of lead body 72 such that electrode 74 is recessed relative to lead body 72. Recessed electrode 74 may aid in limiting the distance a stimulation field extends from an outer diameter of lead body 72 in radial direction 78 perpendicular to the longitudinal axis of lead body 72 relative to an electrode having a diameter D2 equal to diameter D1 of lead body 72. The distance a stimulation field extends from an outer diameter of lead body 22 in radial direction 28 perpendicular to the longitudinal axis of lead body 22 may also be referred to as the depth of the stimulation field. The recessed electrode 74 draws the stimulation field closer to the longitudinal axis of lead body 72. In this manner, the relationship between diameter D2 of electrode 74 and D1 of lead body 72 may aid in controlling the depth of the stimulation field.

Shield 80 is positioned on an outer surface of recessed ring electrode 74 such that shield 80 is substantially flush with lead body 72. This allows lead 80 to be isodiametric throughout the length of lead body 72, which may be helpful in preventing thrombosis. Allowing lead 80 to be isodiametric throughout the length of lead body 72 may also make implantation of lead 80 easier.

FIG. 7 is a side view of an example of a distal end of a lead 90. Like lead 70, lead 90 is also substantially similar to lead 40 of FIGS. 5A and 5B but includes a protruded ring electrode 94. Lead 90 includes a lead body 92 and electrodes 94 and 96A-96C. Electrodes 96A-96C may be substantially similar to electrodes 46A-46C of lead 40 and may be arranged in a similar configuration.

Electrode 94 protrudes relative to lead body 92. More particularly, the diameter D4 of electrode 94 is larger than the diameter D3 of lead body 92 such that electrode 84 protrudes relative to lead body 92. Protruded electrode 94 may aid in increasing the distance a stimulation field extends from an outer diameter of lead body 92 in radial direction 98 perpendicular to the longitudinal axis of lead body 92 relative to an electrode having a diameter D4 equal to diameter D3 of lead body 92. The protruded electrode 94 extends the stimulation field farther from the longitudinal axis of lead body 92. In this manner, the relationship between diameter D4 of electrode 94 and D3 of lead body 92 may aid in controlling the depth of the stimulation field. A stimulation field with increased depth may be useful in delivering stimulation to a target stimulation site further from lead body 92 than reachable if the diameter D4 of electrode 94 equaled the diameter D3 of lead body 92.

Recessed and protruded electrodes are described in further detail in commonly-assigned U.S. Utility patent application Ser. No. ______ by Eggen et al., entitled, “STIMULATION FIELD MANAGEMENT” (attorney docket number P0030110.02/1111-006US01), which was filed on the same date as the present disclosure and is hereby incorporated by reference.

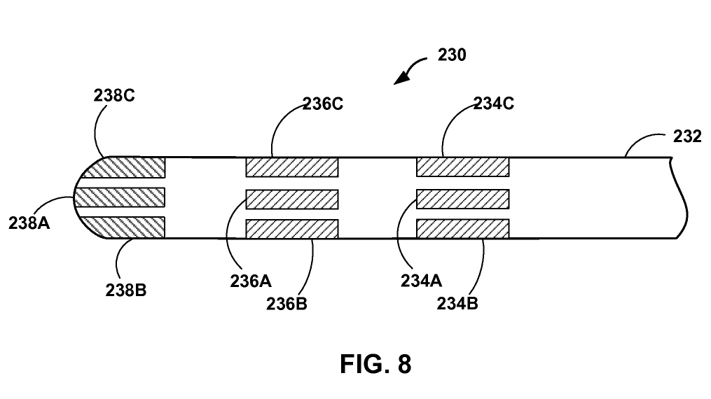

FIG. 8 is a side view of a distal end of another example lead 230 including electrode segments 234A-234B, 236A-236C and 238A-238C at its distal end. Lead 230 is substantially similar to lead 40 of FIGS. 5A and 5B but includes additional electrode segments 234A-234C and 236A-236C axially displaced from electrode segments 238A-238C. Lead 230 includes a lead body 232 and electrodes 234A-234B, 236A-236C, and 238A-238C.

Electrodes 238A-238C may be substantially similar to electrodes 46A-46C of lead 40 and may be arranged in a similar configuration. For example, a cross-sectional view of electrodes 238A-238C may be substantially similar to the cross-sectional view of electrode 46A-46C illustrated in FIG. 5B. Additionally, both rows of electrode segments 236A-236C and 234A-234C may have cross-sections substantially similar to the example illustrated in FIG. 5B. However, the configuration, number, and type of electrodes illustrated in and described with respect to FIG. 8 are merely exemplary. In other examples, lead 230 may include any number of rows of electrode segments, any number of electrode segments per row, and any cross-sectional configuration. Lead 230 may also include electrode segments positioned at various radial and axial positions of lead insulative body 232 such that the electrode segments do not form rows.

An IMD (e.g., IMD 12 of FIG. 1) may configure one of electrode segments 234A-234C, 236A-236C, and 238A-238C as a cathode and two adjacent electrode segments as anodes. As one example, IMD 12 may configure electrode segment 236A as a cathode and electrode segments 236B and 238A as anodes. Electrode segment 236B (the first anode) is located at a radial position adjacent to electrode segment 236A (the cathode) and the same axial position as electrode segment 236A (the cathode). Electrode segment 238A (the second anode) is located at the same radial position as electrode segment 236A (the cathode) and an axial position adjacent to electrode segment 236A (the cathode). In this manner, the electrical field may be constrained from extending beyond electrode segments 236B and 238A (the anodes). For example, the electrical field may not extend transversely outward from the portion of lead body 232 containing electrode segment 236B. Additionally, the electrical field may not extend past electrode segment 238A such that the most distal point of the electrical field may be located at electrode segment 238A. The anode and cathode configuration may be based on the location of a target tissue site and/or an undesirable stimulation site.

As another example, IMD 12 may configure electrode segment 236A as a cathode and electrode segments 234A and 238A as anodes. Electrode segments 234A and 238A (the anodes) are located at the same radial position as electrode segment 236A (the cathode) and axial positions adjacent to electrode segment 236A (the cathode). In this manner, the electrical field may be constrained from extending beyond electrode segments 234A and 238A (the anodes). For example, the electrical field may not extend more distal than electrode segment 238A or more proximal than electrode segment 234A. Such an anodal shielding configuration may be used to limit the length of the electrical field along the length of lead body 232, e.g., to constrain the electrical field in a longitudinal direction.

Other anodal shielding configurations may use two or more electrode segments at one or more radial position of lead 230 and one or more axial position of lead 230. For example, in some examples, three or more electrode segments 234, 236, 238 at various axial or radial positions relative to a cathode may be activated to substantially surround the cathode, e.g., four more adjacent electrode segments forming a square, diamond, or other geometric shaped “box” around the cathode may be activated as anodes to constrain the resulting electrical field. Any anodal shielding configuration including a cathode and two or more adjacent anodes may be utilized to direct the electrical field toward a target tissue site and/or away from an undesirable site.

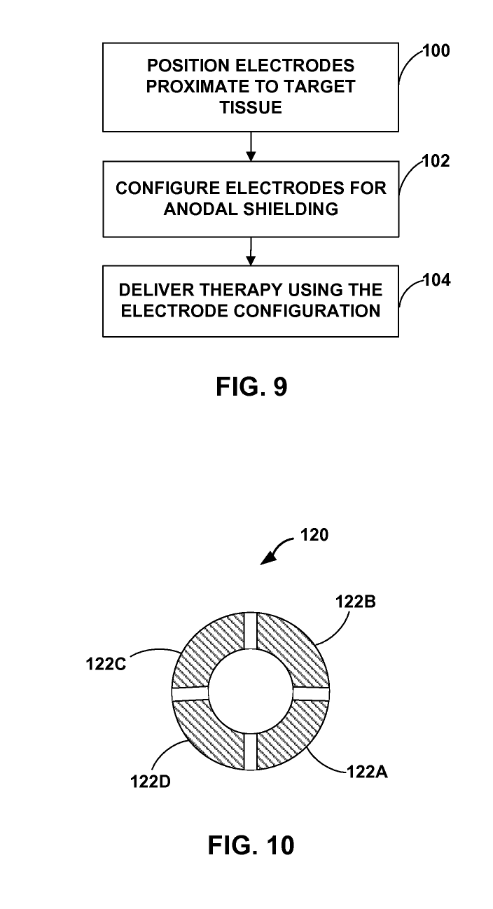

FIG. 9 is a flowchart illustrating a method of delivering stimulation therapy using an anodal shielding configuration. While the process shown in FIG. 9 is described with respect to lead 40 of FIGS. 5A and 5B, in other examples, the lead may be, for example, any one of leads 14, 16, 20, 70, 90 or 230 of FIGS. 1, 4A-4D, 6, 7 and 8. In addition, the process shown in FIG. 9 may be used to implant any suitable lead including electrode segments.

Electrode segments 46A-46C are positioned proximate to a target tissue (100). In some examples, electrode segments 46A-46C may be positioned proximate to the left ventricle or the vagus nerve of patient 18 (FIG. 1). As described previously, lead 40 may include one or more markers (e.g., radiographic and/or visible markers) to aid in positioning electrode segments 46A-46C. For example, markers may provide an indication of the position of electrode segments 46A-46C. In some examples, one or more of electrode segments 46A-46C may be made of platinum iridium or another material that is detectable via imaging techniques. In this manner, one or more of electrode segments 46A-46C may be markers. Electrode segments 46A-46C may be positioned based on the location of one or more markers.

Once electrode segments 46A-46C have been positioned, an IMD (e.g., IMD 12 of FIG. 1) configures the electrodes for anodal shielding (102). In general, an anodal shielding configuration includes a cathode with two adjacent anodes (e.g., on opposite sides of the cathode). As one example, IMD 12 (FIG. 1) may configure electrode segment 46B as a cathode and electrode segments 46A and 46C on opposite sides of electrode segments 46B as anodes. IMD 12 may configure electrode segments as controlled by programmer 19, a user of the programmer, and/or programs stored in memory 202 of the IMD or 222 of the programmer. Once electrode segments 46A-46C are configured for anodal shielding, therapy is delivered to the target tissue (e.g., the left ventricle or vagus nerve) of patient 18 (FIG. 1) via electrode segments 46A-46C (104).

### Experimental Results

A single subject swine experiment was conducted using a quadpole 5.5 French segmented lead consisting of polymer tip with four electrically independent electrodes each having a surface area of 2.2 square millimeters (mm2). FIG. 10 is a cross-section of the segmented lead 120 illustrating the four electrode segments 122A-122D. An over the lead body helix was used to actively fixate the lead in the vein to eliminate rotational and lateral changes in lead position during the study.

The segmented lead 120 was positioned for LV stimulation. Stimulation was delivered using both a unipolar mode and an anodal shielding configuration. The unipolar pacing mode utilized three of electrode segments 122A-122D as cathodes and a RV coil on a second lead implanted in the right ventricle as an anode. The anodal shielding configuration utilized one of the tip electrode segments 122A-122D as a cathode and two of tip electrode segments 122A-122D on opposite sides of the cathode as anodes. A pacing threshold and phrenic nerve stimulation threshold was measured for both the unipolar mode and anodal shielding configuration. Table 1 illustrates the pacing and phrenic nerve stimulation thresholds measured using the unipolar mode, and Table 2 illustrates the pacing and phrenic nerve stimulation thresholds measured using the anodal shielding configuration. A-D correspond to electrode segments 122A-122D, respectively, and greater than 10 volts (>10 V) indicates that capture was not obtained as a maximum output of 10 V.

TABLE 1
Unipolar Mode

| Cathodes | Pacing Threshold (V) | Phrenic Threshold (V) |
|---|---|---|
| A, B, C | 3.5 | 1.7 |
| B, C, D | 3.5 | 1.6 |
| C, D, A | 6 | 1.5 |
| D, A, B | 3.5 | 1.6 |

TABLE 2
Anodal Shielding Configuration

| Anode, Cathode, Anode | Pacing Threshold (V) | Phrenic Threshold (V) |
|---|---|---|
| A, B, C | 5.3 | >10 |
| B, C, D | >10 | >10 |
| C, D, A | >10 | 7 |
| D, A, B | >10 | >10 |

The phrenic nerve stimulation threshold was higher using the anodal shielding configuration than the unipolar configuration. For each anodal shielding configuration tested, the stimulation field was rotated 90 degrees. When the field was pointed at the myocardium, myocardial capture was achieved. When the field was pointed at the phrenic nerve, phrenic capture was accomplished. For the other two cases neither phrenic nerve nor the myocardium was captured.

Another experiment was conducted using the same type of lead in a canine great vein. Stimulation was delivered using both unipolar and anodal shielding configurations. In unipolar mode, one or more of electrode segments 122A-122D were configured as cathodes and a ring electrode on another lead (i.e., a CapSureFix® Novus Lead, Model 5076 commercially available from Medtronic, Inc. of Minneapolis, Minn.) positioned in the right ventricle was set as the anode. For anodal shielding configurations, one of electrode segments 122A-122D was set as a cathode and two of electrode segments 122A-122D adjacent opposite sides of the cathode were set as anodes. Thresholds for both pacing and phrenic nerve stimulation were measured as well as the electrode impedances.

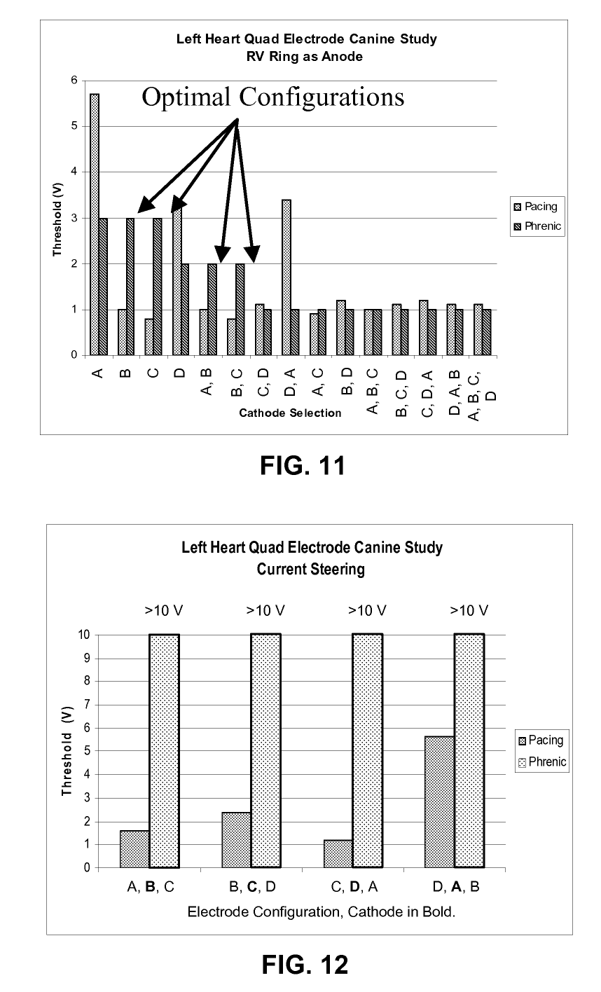

FIG. 11 illustrates the pacing and phrenic nerve stimulation thresholds measured using the unipolar mode, and FIG. 12 illustrates the pacing and phrenic nerve stimulation thresholds measured using the anodal shielding configurations. A-D correspond to electrode segments 122A-122D, respectively, and greater than 10 volts (>10 V) indicates that capture was not obtained as a maximum output of 10 V. During unipolar pacing, optimal electrode configurations existed where the pacing capture threshold was below the phrenic nerve capture threshold. This benefit diminished as the electrode configuration more closely resembled a “ring-like” geometry where all segments were cathodes. Using anodal shielding, phrenic nerve stimulation was avoided at maximum device output despite the lead being the same location as when the unipolar stimulation was delivered.

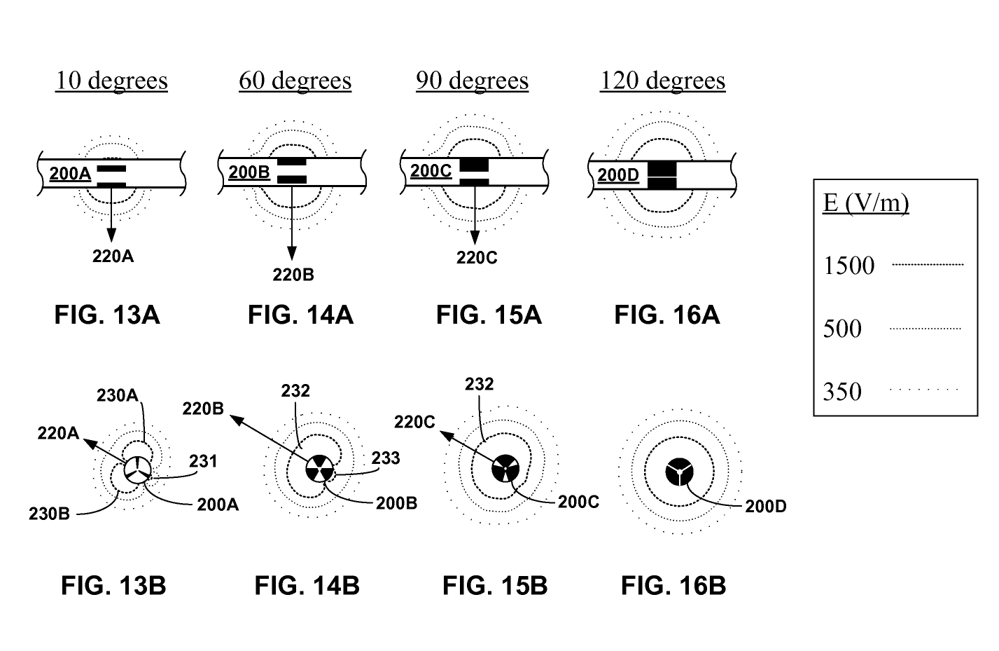

FIGS. 13A-16B illustrate side and cross-section views of example leads having electrode segments, and example electrical fields produced when two of the electrode segments are charged. Specifically, FIGS. 13A, 14A, 15A and 16A illustrate cross-sectional views of leads 200A, 200B, 200C and 200D, respectively and also illustrate the corresponding electrical fields. FIGS. 13B, 14B, 15B and 16B side views of leads 200A, 200B, 200C and 200D respectively and likewise illustrate the corresponding electrical fields. While leads 200A-200D are each shown with a single segmented electrode consisting of three electrode segments, leads 200A-200D may also have additional electrodes and different examples may comprise segmented electrodes including more than three electrode segments as previously described herein.

Two adjacent electrode segments on each of leads 200A-200D are configured as cathodes, whereas the other electrode segment is configured to be electrically isolated. The electric fields shown assume that the anode is positioned at a distant location relative to the cathodes. As examples, the anode could be, e.g., a metallic housing of an IMD including a simulation generator used to charge the electrode segments configured as cathodes, or a ring electrode or other anode located proximally on the lead relative of the illustrated electrode segments. The anode may have a larger surface area than the combined surface area of the electrode segments activated as cathodes.

Lead 200A includes three equally spaced electrode segments, each segment covering an arc of the circumference of the lead body of 10 degrees. The electric field includes a field centroid along vector 220A. However, the relatively small arc of the electrode segments in lead 200A results in two separate areas 230A and 230B of high field concentration. Lead 200A also provides an incidental area 231 of high field concentration resulting from the edge effect of the isolated electrode segment.

Lead 200B includes three equally spaced electrode segments, each segment covering an arc of the circumference of the lead body of 60 degrees. The electric field includes a field centroid along vector 220B. Lead 200B also provides a single area 232 of high field concentration centered along vector 220B. Lead 200B also provides an incidental area 233 of high field concentration resulting from the edge effect of the isolated electrode segment.

Lead 200C includes three equally spaced electrode segments, each segment covering an arc of the circumference of the lead body of 90 degrees. The electric field produces a field centroid along vector 220C. Lead 200C also provides a single area 234 of high field concentration centered along vector 220C. However, relative to lead 200B, the field concentration of lead 200C is less directional as the area of high field concentration 234 extends a further distance from lead 200C in a direction opposite vector 220C than the area of high field concentration 232 extends from lead 200B in a direction opposite vector 220B.

Lead 200D includes three equally spaced electrode segments each covering an arc of the circumference of the lead body of 120 degrees. The three electrode segments of lead 200D provide an electric field that approximates an electric field provided by a single ring electrode.

Because the electrode segments of lead 200D are immediately adjacent each other, each electrode segments has the same voltage potential. This can occur if electrode segments are not separated sufficiently to be electrically isolated from each other. In order to electrically isolate adjacent electrode segments, electrodes segments should cover arcs of no greater than θₘₐₓ, wherein:

$${Θ}_{max} = \frac{360 °}{number\_of \_electrode \_segments} - 10 ° \qquad Equation 1$$

The three electrode segments in leads 200A and 200B cover no more than 180 degrees of the circumference of the lead body at the segmented electrode. For example, the electrode segments of lead 200A cover a total of 30 degrees of the circumference of the lead body at the segmented electrode, and the electrode segments of lead 200B cover a total of 180 degrees of the circumference of the lead body at the segmented electrode, equal to fifty percent of the circumference of the lead body at the segmented electrode. For example, in a segmented electrode consisting of four electrode segments, each electrode segment may cover 45 degrees of the circumference of the lead body at the segmented electrode. Segmented electrodes having more than four electrode segments may also be used.

In other examples, electrode segments may cover between 5 and 92 percent of the circumference of the lead body at the segmented electrode, between 5 and 50 percent of the circumference of the lead body at the segmented electrode, between 25 and 50 percent of the circumference of the lead body at the segmented electrode or between 50 and 75 percent of the circumference of the lead body at the segmented electrode. As an example, a segmented electrode consisting of three electrode segments of 90 degrees each would cover 75 percent of the circumference of the lead body at the segmented electrode.

The electric fields produced using at least two adjacent electrode segments as cathodes may be combined with the previously-described techniques utilizing anodal shielding. A single IMD may optionally configure electrode segments using a single electrode segment as a cathode, using multiple electrode segments as cathodes, as well configuring electrode segments in anodal shielding configuration. An IMD that provides each of these techniques may be able to more successfully direct a stimulation field toward a target site and/or away from an undesirable site.

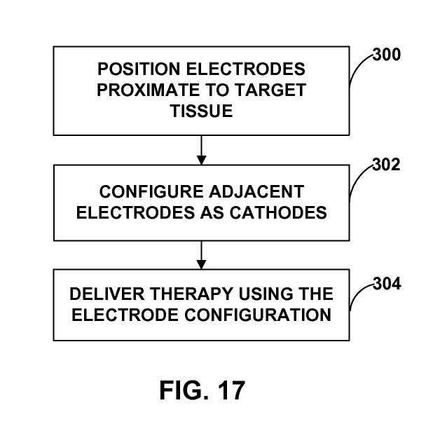

FIG. 17 is a flowchart illustrating a method of delivering stimulation therapy using at least two adjacent electrodes as cathodes. While the process shown in FIG. 17 is described with respect to lead 40 of FIGS. 5A and 5B, in other examples, the lead may be, for example, any one of leads 14, 16, 20, 70, 90 or 230 of FIGS. 1, 4A-4D, 6, 7 and 8. In addition, the process shown in FIG. 17 may be used to implant any suitable lead including electrode segments.

Electrode segments 46A-46C are positioned proximate to a target tissue (300). In some examples, electrode segments 46A-46C may be positioned proximate to the left ventricle or the vagus nerve of patient 18 (FIG. 1). As described previously, lead 40 may include one or more markers (e.g., radiographic and/or visible markers) to aid in positioning electrode segments 46A-46C. For example, markers may provide an indication of the position of electrode segments 46A-46C. In some examples, one or more of electrode segments 46A-46C may be made of platinum iridium or another material that is detectable via imaging techniques. In this manner, one or more of electrode segments 46A-46C may be markers. Electrode segments 46A-46C may be positioned based on the location of one or more markers.

Once electrode segments 46A-46C have been positioned, an IMD (e.g., IMD 12 of FIG. 1) configures at least two of the electrode segments as cathodes and configures at least one additional electrode as being electrically isolated from the cathode electrodes (302). As one example, IMD 12 (FIG. 1) may configure electrode segments 46A and 46B as cathodes and electrically isolate electrode segments 46C and 46D from electrode segments 46A and 46B and the housing of IMD 12. IMD 12 may configure electrode segments as controlled by programmer 19, a user of the programmer, and/or programs stored in memory 202 of the IMD or 222 of the programmer. Once electrode segments 46A-46D are configured, therapy is delivered to the target tissue (e.g., the left ventricle or vagus nerve) of patient 18 (FIG. 1) via electrode segments 46A and 46B (104). For example, the housing of IMD 12 (FIG. 1) may serve as a cathode for the stimulation.

Various examples have been described. However, modifications to the described examples may be made within the spirit of the present disclosure. For example, the described examples include implantable cardiac stimulators, but the described techniques may also be used with external cardiac stimulators. As another example, leads used in conjunction with the techniques described herein may include fixation mechanisms, such as tines that passively secure a lead in an implanted position or a helix located at a distal end of the lead that requires rotation of the lead during implantation to secure the helix to a body tissue. Further, although depicted herein as being located at a distal end of a lead body, in other examples electrode segments capable of being configured as described herein may be located at any axial position of the lead body. These and other examples are within the scope of the following claims.

1. A system comprising:
an implantable electrical stimulation lead configured for intravenous introduction into a vessel proximate to a heart, wherein the lead comprises:
a lead body, and
at least three electrode segments; and
a cardiac stimulator coupled to the electrode segments that configures a first of the electrode segments as a first anode, a second of the electrode segments as a cathode, and a third of the electrode segments as a second anode, and delivers electrical stimulation to the heart via the cathode and first and second anodes.

2. The system of claim 1, further comprising a fourth electrode axially displaced from the first, second, and third electrode segments along a length of the lead.

3. The system of claim 2, wherein the fourth electrode comprises a ring electrode.

4. The system of claim 2, wherein the fourth electrode comprises an electrode segment.

5. The system of claim 2, wherein the fourth electrode comprises an electrode selected from a group consisting of:
an electrode recessed relative to the lead body; and
an electrode that protrudes relative to the lead body.

6. The system of claim 1, wherein a surface area of the first electrode segment and a surface area of the third electrode segment are each at least as large as a surface area of the second electrode segment.

7. The system of claim 1, wherein an insulative material separates each of the first, second, and third electrode segments.

8. The system of claim 1, wherein each of the first, second, and third electrode segments are positioned at substantially the same axial position along a length of the lead.

9. The system of claim 1, wherein at least one of the first, second, and third electrode segments being axially displaced from the others of the first, second, and third electrode segments.

10. The system of claim 1, wherein the first and third electrode segments are positioned on opposite sides of the second electrode segment.

11. The system of claim 1, wherein the lead further comprises at least one additional electrode segment adjacent to the second electrode segment, and the cardiac stimulator configures the at least one additional electrode segment as an additional anode.

12. The system of claim 1, wherein the lead further comprises a plurality of electrode segments, and the cardiac stimulator selects the first, second and third electrode segments from among the plurality of electrode segments.

13. The system of claim 1, wherein the cardiac stimulator delivers at least two different electrical signals via the first, second and third electrode segments substantially simultaneously, and the at least two electrical signals have different amplitudes.

14. The system of claim 1, wherein the cardiac stimulator comprises an implantable cardiac stimulator.

15. The system of claim 14, further comprising an external programmer that sends the implantable cardiac stimulator instructions for configuring the first, second, and third electrode segments.

16. The system of claim 1, wherein the lead includes a marker visible via fluoroscopic imaging to demonstrate the orientation of the lead.

17. A system comprising:
an implantable electrical therapy lead configured for implantation proximate to a vagus nerve of a patient, wherein the lead comprises:
a lead body, and
at least three electrode segments; and
a neurostimulator coupled to the electrode segments that configures a first of the electrode segments as a first anode, a second of the electrode segments as a cathode, and a third of the electrode segments as a second anode, and delivers electrical therapy to the vagus nerve via the cathode and first and second anodes.

18. The system of claim 17, wherein a surface area of the first electrode segment and a surface area of the third electrode segment are each at least as large as a surface area of the second electrode segment.

19. The system of claim 17, wherein each of the first, second, and third electrode segments are positioned at substantially the same axial position along a length of the lead.

20. The system of claim 17, wherein the lead further comprises at least one additional electrode segment adjacent to the second electrode segment, and the cardiac stimulator configures the at least one additional electrode segment as an additional anode.

21. The system of claim 17, wherein the lead further comprises a plurality of electrode segments, and the cardiac stimulator selects the first, second and third electrode segments from among the plurality of electrode segments.

22. The system of claim 17, wherein the lead includes a marker visible via fluoroscopic imaging to demonstrate the orientation of the lead.

23. A method of delivering electrical stimulation to a heart comprising:
configuring a first electrode segment of an implantable electrical stimulation lead configured for intravenous introduction into a heart, as a first anode, a second electrode segment of the lead as a cathode, and a third electrode segment of the lead as a second anode; and
delivering at least one electrical stimulation signal to the heart via the first, second, and third electrode segments.

24. The method of claim 23, wherein delivering the stimulation signal comprises delivering the stimulation signal to a left ventricle of the patient via the first, second, and third electrode segments.

25. The method of claim 23, further comprising positioning the second electrode proximate to a target stimulation site.

26. The method of claim 25, wherein positioning the second electrode comprises positioning the second electrode based on a location of a marker.

27. The method of claim 26, wherein the second electrode comprises the marker.

28. The method of claim 23, wherein configuring the first, second and third electrodes comprises directing the stimulation toward myocardium.

29. The method of claim 23, wherein configuring the first, second and third electrodes comprises directing the stimulation away from a phrenic nerve.

30. The method of claim 23, wherein delivering the at least one electrical stimulation signal comprises delivering pacing pulses configured to capture the heart.

31. A method of delivering electrical therapy to a vagus nerve of a patient comprising:
configuring a first electrode segment of an implantable electrical therapy lead configured for implantation proximate to the vagus nerve, as a first anode, a second electrode segment of the lead as a cathode, and a third electrode segment of the lead as a second anode; and
delivering at least one electrical therapy signal to the vagus nerve via the first, second, and third electrode segments.

32. The method of claim 31, further comprising positioning the second electrode proximate to a target therapy site.

33. The method of claim 32, wherein positioning the second electrode comprises positioning the second electrode based on a location of a marker.

34. A system comprising:
means for configuring a first electrode segment of an implantable electrical stimulation lead as a first anode, a second electrode segment of the lead as a cathode, and a third electrode segment of the lead as a second anode; and
means for delivering a stimulation signal via the first, second, and third electrode segments to one of a group consisting of:
a heart; and
a vagus nerve of a patient.

35. A system comprising:
an implantable electrical stimulation lead configured for intravenous introduction into a vessel proximate to a heart, wherein the lead comprises:
a lead body,
a segmented electrode including at least three electrode segments, and
insulative material between the at least three electrode segments at an outer circumference of the lead body at the segmented electrode,
wherein the at least three electrode segments are spaced apart circumferentially and separated by the insulative material such that the at least three electrode segments cover no more than about 270 degrees of the outer circumference of the lead body at the segmented electrode; and
a medical device electrically coupled to the electrode segments, wherein the medical device being selected from a group consisting of:
a cardiac stimulator; and
a neurostimulator.

36. The system of claim 35, wherein the at least three electrode segments consist of electrode segments configured to each cover an arc of the circumference of the lead body at the segmented electrode of between about 10 degrees and about 60 degree.

37. A method of delivering electrical stimulation to a heart comprising:
configuring at least two adjacent electrode segments of a segmented electrode as cathodes, wherein the segmented electrode being included in an implantable electrical stimulation lead configured for intravenous introduction into a heart;
configuring at least a third electrode segment of the segmented electrode to be electrically isolated from the cathodes; and
delivering at least one electrical stimulation signal to the heart via the at least two adjacent electrode segments,
wherein the electrode segments of the segmented electrode are spaced apart circumferentially and separated by an insulative material such that the electrode segments of the segmented electrode cover no more than about 270 degrees of an outer circumference of the lead body at the segmented electrode.

38. The method of claim 37, wherein delivering the stimulation signal comprises delivering the stimulation signal to a left ventricle of the patient.

39. The method of claim 37, wherein configuring the at least two adjacent electrode segments comprises directing the stimulation away from a phrenic nerve.

40. A method of delivering electrical therapy to a vagus nerve of a patient comprising:
configuring at least two adjacent electrode segments of an implantable electrical stimulation lead configured for intravenous introduction into a heart as cathodes;
configuring at least a third electrode segment of the lead to be electrically isolated from the cathodes; and
delivering at least one electrical therapy signal to the vagus nerve via the at least two adjacent electrode segments,
wherein the electrode segments of the segmented electrode are spaced apart circumferentially and separated by an insulative material such that the electrode segments of the segmented electrode cover no more than about 270 degrees of an outer circumference of the lead body at the segmented electrode.

41. The method of claim 40, further comprising positioning the at least two adjacent electrode segments proximate to a target therapy site including the vagus nerve.
